(19) United States
(12) Patent Application Publication
(10) Pub. No.: US 2008/0200489 A1
(43) Pub. Date: Aug. 21, 2008

(54) Combination of Organic Compounds

(76) Inventors: Peter Wisdom Atadja, Acton, MA (US); Kapil N. Bhalla, Tampa, FL (US); Paul W. Manley, Arleshiem (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/063,172

(22) PCT Filed: Aug. 10, 2006
(86) PCT No.: PCT/US2006/031563

§ 371 Date: Feb. 7, 2008

Related U.S. Application Data
(60) Provisional application No. 60/707,436, filed on Aug. 11, 2005.

(51) Int. Cl. A61K 31/506; A61P 35/02

(52) U.S. Cl. 514/275

(57) ABSTRACT

The invention provides a pharmaceutical combination comprising:
a) a pyrimidylaminobenzamide compound; and
b) an HDAC inhibitor compound,
   and a method for treating or preventing a proliferative disease using such a combination.

The present invention relates to a pharmaceutical combination comprising a pyrimidylaminobenzamide compound and a histone deacetylase inhibitor, and the uses of such a combination, e.g., in proliferative diseases, e.g., tumors, myelomas, leukemias, psoriasis, restenosis, sclerodermitis and fibrosis.

In spite of numerous treatment options for proliferative disease patients, there remains a need for effective and safe antiproliferative agents and a need for their preferential use in combination therapy.

Reversible acetylation of histones is a major regulator of gene expression that acts by altering accessibility of transcription factors to DNA. In normal cells, histone deacetylase (HDAC) and histone acetyltrasferase together control the level of acetylation of histones to maintain a balance. Inhibition of HDAC results in the accumulation of hyperacetylated histones, which results in a variety of cellular responses. Inhibitors of HDAC have been studied for their therapeutic effects on cancer cells. Recent developments in the field of HDAC inhibitors research have provided active compounds, both highly efficacious and stable, that are suitable for treating tumors.

### SUMMARY OF THE INVENTION

It has now been found that a combination comprising at least one pyrimidylaminobenzamide compound and an HDAC inhibitor, e.g., as defined below, has a beneficial effect on proliferative diseases, e.g., tumors, myelomas, leukemias, psoriasis, restenosis, sclerodermitis and fibrosis.

### DETAILED DESCRIPTION OF THE INVENTION

### Pyrimidylaminobenzamide Compounds:

The present invention relates to the use of pyrimidylaminobenzamide compounds of formula (I):

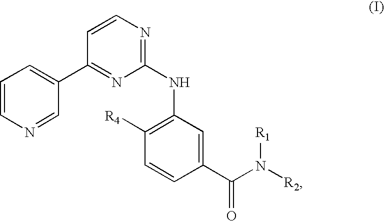

wherein
R₁ represents hydrogen, lower alkyl, lower alkoxy-lower alkyl, acyloxy-lower alkyl, carboxy-lower alkyl, lower alkoxycarbonyl-lower alkyl, or phenyl-lower alkyl;
R₂ represents hydrogen, lower alkyl, optionally substituted by one or more identical or different radicals R₃, cycloalkyl, benzcycloalkyl, heterocyclyl, an aryl group, or a mono- or bicyclic heteroaryl group comprising zero, one, two or three ring nitrogen atoms and zero or one oxygen atom and zero or one sulfur atom, which groups in each case are unsubstituted or mono- or polysubstituted; and R₃ represents hydroxy, lower alkoxy, acyloxy, carboxy, lower alkoxycarbonyl, carbamoyl, N-mono- or N,N-disubstituted carbamoyl, amino, mono- or disubstituted amino, cycloalkyl, heterocyclyl, an aryl group, or a mono- or bicyclic heteroaryl group comprising zero, one, two or three ring nitrogen atoms and zero or one oxygen atom and zero or one sulfur atom, which groups in each case are unsubstituted or mono- or polysubstituted, or
R₁ and R₂ together represent alkylene with four, five or six carbon atoms optionally mono- or disubstituted by lower alkyl, cycloalkyl, heterocyclyl, phenyl, hydroxy, lower alkoxy, amino, mono- or disubstituted amino, oxo, pyridyl, pyrazinyl or pyrimidinyl; benzalkylene with four or five carbon atoms; oxaalkylene with one oxygen and three or four carbon atoms; or azaalkylene with one nitrogen and three or four carbon atoms wherein nitrogen is unsubstituted or substituted by lower alkyl, phenyl-lower alkyl, lower alkoxycarbonyl-lower alkyl, carboxy-lower alkyl, carbamoyl-lower alkyl, N-mono- or N,N-disubstituted carbamoyl-lower alkyl, cycloalkyl, lower alkoxycarbonyl, carboxy, phenyl, substituted phenyl, pyridinyl, pyrimidinyl, or pyrazinyl;
R₄ represents hydrogen, lower alkyl, or halogen;
   and a N-oxide or a pharmaceutically acceptable salt of such a compound for the preparation of a pharmaceutical composition for the treatment of kinase dependent diseases.

The general terms used hereinbefore and hereinafter preferably have within the context of this disclosure the following meanings, unless otherwise indicated:

The prefix “lower” denotes a radical having up to and including a maximum of 7, especially up to and including a maximum of 4 carbon atoms, the radicals in question being either linear or branched with single or multiple branching.

Where the plural form is used for compounds, salts, and the like, this is taken to mean also a single compound, salt, or the like.

Any asymmetric carbon atoms may be present in the (R)—, (S)— or (R,S)-configuration, preferably in the (R)— or (S)-configuration. The compounds may thus be present as mixtures of isomers or as pure isomers, preferably as enantiomer-pure diastereomers.

The invention relates also to possible tautomers of the compounds of formula (I).

Lower alkyl is preferably alkyl with from and including 1 up to and including 7, preferably from and including 1 to and including 4, and is linear or branched; preferably, lower alkyl is butyl, such as n-butyl, sec-butyl, isobutyl, tert-butyl, propyl, such as n-propyl or isopropyl, ethyl or methyl. Preferably lower alkyl is methyl, propyl or tert-butyl.

Lower acyl is preferably formyl or lower alkylcarbonyl, in particular, acetyl.

An aryl group is an aromatic radical which is bound to the molecule via a bond located at an aromatic ring carbon atom of the radical. In a preferred embodiment, aryl is an aromatic radical having 6 to 14 carbon atoms, especially phenyl, naphthyl, tetrahydronaphthyl, fluorenyl or phenanthrenyl, and is unsubstituted or substituted by one or more, preferably up to three, especially one or two substituents, especially selected from amino, mono- or disubstituted amino, halogen, lower alkyl, substituted lower alkyl, lower alkenyl, lower alkynyl, phenyl, hydroxy, etherified or esterified hydroxy, nitro, cyano, carboxy, esterified carboxy, alkanoyl, benzoyl, carbamoyl, N-mono- or N,N-disubstituted carbamoyl, amidino, guanidino, ureido, mercapto, sulfo, lower alkylthio, phenylthio, phenyl-lower alkylthio, lower alkylphenylthio, lower alkylsulfinyl, phenylsulfinyl, phenyl-lower alkylsulfinyl, lower alkylphenylsulfinyl, lower alkylsulfonyl, phenylsulfonyl, phenyl-lower alkylsulfonyl, lower alkylphenylsulfonyl, halogen-lower alkylmercapto, halogen-lower alkylsulfonyl, such as especially trifluoromethanesulfonyl, dihydroxybora (—B(OH)₂), heterocyclyl, a mono- or bicyclic heteroaryl group and lower alkylene dioxy bound at adjacent C-atoms of the ring, such as methylene dioxy. Aryl is more preferably phenyl, naphthyl or tetrahydronaphthyl, which in each case is either unsubstituted or independently substituted by one or two substituents selected from the group comprising halogen, especially fluorine, chlorine, or bromine; hydroxy; hydroxy etherified by lower alkyl, e.g., by methyl, by halogen-lower alkyl, e.g. trifluoromethyl, or by phenyl; lower alkylene dioxy bound to two adjacent C-atoms, e.g., methylenedioxy, lower alkyl, e.g., methyl or propyl; halogen-lower alkyl, e.g. trifluoromethyl; hydroxy-lower alkyl, e.g., hydroxymethyl or 2-hydroxy-2-propyl; lower alkoxy-lower alkyl; e.g., methoxymethyl or 2-methoxyethyl; lower alkoxycarbonyl-lower alkyl, e.g., methoxycarbonylmethyl; lower alkynyl, such as 1-propynyl; esterified carboxy, especially lower alkoxycarbonyl, e.g., methoxycarbonyl, n-propoxy carbonyl or iso-propoxy carbonyl; N-mono-substituted carbamoyl, in particular carbamoyl monosubstituted by lower alkyl, e.g., methyl, n-propyl or iso-propyl; amino; lower alkylamino, e.g., methylamino; di-lower alkylamino, e.g., dimethylamino or diethylamino; lower alkylene-amino, e.g., pyrrolidino or piperidino; lower oxaalkylene-amino, e.g., morpholino, lower azaalkylene-amino, e.g., piperazino, acylamino, e.g., acetylamino or benzoylamino; lower alkylsulfonyl, e.g., methylsulfonyl; sulfamoyl; or phenylsulfonyl.

A cycloalkyl group is preferably cyclopropyl, cyclopentyl, cyclohexyl or cycloheptyl, and may be unsubstituted or substituted by one or more, especially one or two, substituents selected from the group defined above as substituents for aryl, most preferably by lower alkyl, such as methyl, lower alkoxy, such as methoxy or ethoxy, or hydroxy, and further by oxo or fused to a benzo ring, such as in benzcyclopentyl or benzcyclohexyl.

Substituted alkyl is alkyl as last defined, especially lower alkyl, preferably methyl; where one or more, especially up to three, substituents may be present, primarily from the group selected from halogen, especially fluorine, amino, N-lower alkylamino, N,N-di-lower alkylamino, N-lower alkanoylamino, hydroxy, cyano, carboxy, lower alkoxycarbonyl, and phenyl-lower alkoxycarbonyl. Trifluoromethyl is especially preferred.

Mono- or disubstituted amino is especially amino substituted by one or two radicals selected independently of one another from lower alkyl, such as methyl; hydroxy-lower alkyl, such as 2-hydroxyethyl; lower alkoxy lower alkyl, such as methoxy ethyl; phenyl-lower alkyl, such as benzyl or 2-phenylethyl; lower alkanoyl, such as acetyl; benzoyl; substituted benzoyl, wherein the phenyl radical is especially substituted by one or more, preferably one or two, substituents selected from nitro, amino, halogen, N-lower alkylamino, N,N-di-lower alkylamino, hydroxy, cyano, carboxy, lower alkoxycarbonyl, lower alkanoyl, and carbamoyl; and phenyl-lower alkoxycarbonyl, wherein the phenyl radical is unsubstituted or especially substituted by one or more, preferably one or two, substituents selected from nitro, amino, halogen, N-lower alkylamino, N,N-di-lower alkylamino, hydroxy, cyano, carboxy, lower alkoxycarbonyl, lower alkanoyl, and carbamoyl; and is preferably N-lower alkylamino, such as N-methylamino, hydroxy-lower alkylamino, such as 2-hydroxyethylamino or 2-hydroxypropyl, lower alkoxy lower alkyl, such as methoxy ethyl, phenyl-lower alkylamino, such as benzylamino, N,N-di-lower alkylamino, N-phenyl-lower alkyl-N-lower alkylamino, N,N-di-lower alkylphenylamino, lower alkanoylamino, such as acetylamino, or a substituent selected from the group comprising benzoylamino and phenyl-lower alkoxycarbonylamino, wherein the phenyl radical in each case is unsubstituted or especially substituted by nitro or amino, or also by halogen, amino, N-lower alkylamino, N,N-di-lower alkylamino, hydroxy, cyano, carboxy, lower alkoxycarbonyl, lower alkanoyl, carbamoyl or aminocarbonylamino. Disubstituted amino is also lower alkylene-amino, e.g., pyrrolidino, 2-oxopyrrolidino or piperidino; lower oxaalkylene-amino, e.g., morpholino, or lower azaalkylene-amino, e.g., piperazino or N-substituted piperazino, such as N-methylpiperazino or N-methoxycarbonylpiperazino.

Halogen is especially fluorine, chlorine, bromine, or iodine, especially fluorine, chlorine, or bromine.

Etherified hydroxy is especially C₈-C₂₀alkyloxy, such as n-decyloxy, lower alkoxy (preferred), such as methoxy, ethoxy, isopropyloxy, or tert-butyloxy, phenyl-lower alkoxy, such as benzyloxy, phenyloxy, halogen-lower alkoxy, such as trifluoromethoxy, 2,2,2-trifluoroethoxy or 1,1,2,2-tetrafluoroethoxy, or lower alkoxy which is substituted by mono- or bicyclic heteroaryl comprising one or two nitrogen atoms, preferably lower alkoxy which is substituted by imidazolyl, such as 1H-imidazol-1-yl, pyrrolyl, benzimidazolyl, such as 1-benzimidazolyl, pyridyl, especially 2-, 3- or 4-pyridyl, pyrimidinyl, especially 2-pyrimidinyl, pyrazinyl, isoquinolinyl, especially 3-isoquinolinyl, quinolinyl, indolyl or thiazolyl.

Esterified hydroxy is especially lower alkanoyloxy, benzoyloxy, lower alkoxycarbonyloxy, such as tert-butoxycarbonyloxy, or phenyl-lower alkoxycarbonyloxy, such as benzyloxycarbonyloxy.

Esterified carboxy is especially lower alkoxycarbonyl, such as tert-butoxycarbonyl, iso-propoxycarbonyl, methoxycarbonyl or ethoxycarbonyl, phenyl-lower alkoxycarbonyl, or phenyloxycarbonyl.

Alkanoyl is primarily alkylcarbonyl, especially lower alkanoyl, e.g., acetyl.

N-Mono- or N,N-disubstituted carbamoyl is especially substituted by one or two substituents independently selected from lower alkyl, phenyl-lower alkyl and hydroxy-lower alkyl, or lower alkylene, oxa-lower alkylene or aza-lower alkylene optionally substituted at the terminal nitrogen atom.

A mono- or bicyclic heteroaryl group comprising zero, one, two or three ring nitrogen atoms and zero or one oxygen atom and zero or one sulfur atom, which groups in each case are unsubstituted or mono- or polysubstituted, refers to a heterocyclic moiety that is unsaturated in the ring binding the heteroaryl radical to the rest of the molecule in formula (I) and is preferably a ring, where in the binding ring, but optionally also in any annealed ring, at least one carbon atom is replaced by a heteroatom selected from the group consisting of nitrogen, oxygen and sulfur; where the binding ring preferably has 5 to 12, more preferably 5 or 6 ring atoms; and which may be unsubstituted or substituted by one or more, especially one or two, substituents selected from the group defined above as substituents for aryl, most preferably by lower alkyl, such as methyl, lower alkoxy, such as methoxy or ethoxy, or hydroxy. Preferably the mono- or bicyclic heteroaryl group is selected from 2H-pyrrolyl, pyrrolyl, imidazolyl, benzimidazolyl, pyrazolyl, indazolyl, purinyl, pyridyl, pyrazinyl, pyrimidinyl, pyridazinyl, 4H-quinolizinyl, isoquinolyl, quinolyl, phthalazinyl, naphthyridinyl, quinoxalyl, quinazolinyl, quinnolinyl, pteridinyl, indolizinyl, 3H-indolyl, indolyl, isoindolyl, oxazolyl, isoxazolyl, thiazolyl, isothiazolyl, triazolyl, tetrazolyl, furazanyl, benzo[d]pyrazolyl, thienyl and furanyl. More preferably the mono- or bicyclic heteroaryl group is selected from the group consisting of pyrrolyl, imidazolyl, such as 1H-imidazol-1-yl, benzimidazolyl, such as 1-benzimidazolyl, indazolyl, especially 5-indazolyl, pyridyl, especially 2-, 3- or 4-pyridyl, pyrimidinyl, especially 2-pyrimidinyl, pyrazinyl, isoquinolinyl, especially 3-isoquinolinyl, quinolinyl, especially 4- or 8-quinolinyl, indolyl, especially 3-indolyl, thiazolyl, benzo[d]pyrazolyl, thienyl, and furanyl. In one preferred embodiment of the invention the pyridyl radical is substituted by hydroxy in ortho position to the nitrogen atom and hence exists at least partially in the form of the corresponding tautomer which is pyridin-(1H)2-one. In another preferred embodiment, the pyrimidinyl radical is substituted by hydroxy both in position 2 and 4 and hence exists in several tautomeric forms, e.g. as pyrimidine-(1H, 3H)2,4-dione.

Heterocyclyl is especially a five, six or seven-membered heterocyclic system with one or two heteroatoms selected from the group comprising nitrogen, oxygen, and sulfur, which may be unsaturated or wholly or partly saturated, and is unsubstituted or substituted especially by lower alkyl, such as methyl, phenyl-lower alkyl, such as benzyl, oxo, or heteroaryl, such as 2-piperazinyl; heterocyclyl is especially 2- or 3-pyrrolidinyl, 2-oxo-5-pyrrolidinyl, piperidinyl, N-benzyl-4-piperidinyl, N-lower alkyl-4-piperidinyl, N-lower alkyl-piperazinyl, morpholinyl, e.g. 2- or 3-morpholinyl, 2-oxo-1H-azepin-3-yl, 2-tetrahydrofuranyl, or 2-methyl-1,3-dioxolan-2-yl.

Salts are especially the pharmaceutically acceptable salts of compounds of formula (I).

Such salts are formed, for example, as acid addition salts, preferably with organic or inorganic acids, from compounds of formula (I) with a basic nitrogen atom, especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

In the presence of negatively charged radicals, such as carboxy or sulfo, salts may also be formed with bases, e.g., metal or ammonium salts, such as alkali metal or alkaline earth metal salts, for example, sodium, potassium, magnesium or calcium salts, or ammonium salts with ammonia or suitable organic amines, such as tertiary monoamines, for example, triethylamine or tri(2-hydroxyethyl)amine, or heterocyclic bases, for example, N-ethyl-piperidine or N,N′-dimethylpiperazine.

When a basic group and an acid group are present in the same molecule, a compound of formula (I) may also form internal salts.

For isolation or purification purposes it is also possible to use pharmaceutically unacceptable salts, for example, picrates or perchlorates. For therapeutic use, only pharmaceutically acceptable salts or free compounds are employed (where applicable in the form of pharmaceutical preparations), and these are therefore preferred.

In view of the close relationship between the novel compounds in free form and those in the form of their salts, including those salts that can be used as intermediates, for example, in the purification or identification of the novel compounds, any reference to the free compounds hereinbefore and hereinafter is to be understood as referring also to the corresponding salts, as appropriate and expedient.

Compounds within the scope of formula (I) and the process for their manufacture are disclosed in WO 04/005281 published on Jan. 15, 2004 which is hereby incorporated into the present application by reference. A preferred compound is 4-methyl-3-[[4-(3-pyridinyl)-2-pyrimidinyl]amino]-N-[5-(4-methyl-1H-imidazol-1-yl)-3-(trifluoromethyl)phenyl]benzamide which has the structure (II), hereinafter “Compound (II)”:

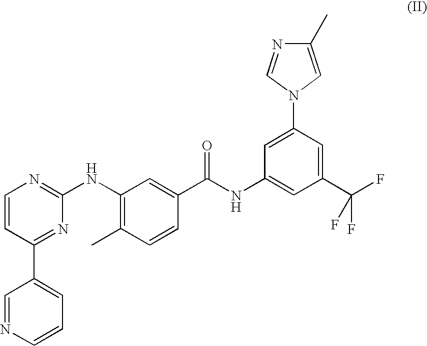

### The HADC Inhibitor Compounds

HDAC compounds of particular interest for use in the inventive combination are hydroxamate compounds described by the formula (III):

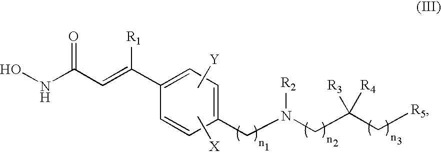

wherein
R₁ is H; halo; or a straight-chain C₁-C₆alkyl, especially methyl, ethyl or n-propyl, which methyl, ethyl and n-propyl substituents are unsubstituted or substituted by one or more substituents described below for alkyl substituents;
R₂ is selected from H; C₁-C₁₀alkyl, preferably C₁-C₆alkyl, e.g., methyl, ethyl or —CH₂CH₂—OH; C₄-C₉cycloalkyl; C₄-C₉heterocycloalkyl; C₄-C₉heterocycloalkylalkyl; cycloalkylalkyl, e.g., cyclopropylmethyl; aryl; heteroaryl; arylalkyl, e.g., benzyl; heteroarylalkyl, e.g., pyridylmethyl; —(CH₂)ₙC(O)R₆; —(CH₂)ₙOC(O)R₆; amino acyl; HON—C(O)—CH═C(R₁)-aryl-alkyl-; and —(CH₂)ₙR₇;
R₃ and R₄ are the same or different and, independently, H; C₁-C₆alkyl; acyl; or acylamino; or
R₃ and R₄, together with the carbon to which they are bound, represent C═O, C═S or C═NR₈; or
R₂, together with the nitrogen to which it is bound, and R₃, together with the carbon to which it is bound, can form a C₄-C₉heterocycloalkyl; a heteroaryl; a polyheteroaryl; a non-aromatic polyheterocycle; or a mixed aryl and non-aryl polyheterocycle ring;
R₅ is selected from H; C₁-C₆alkyl; C₄-C₉cycloalkyl; C₄-C₉heterocycloalkyl; acyl; aryl; heteroaryl; arylalkyl, e.g., benzyl; heteroarylalkyl, e.g., pyridylmethyl; aromatic polycycles; non-aromatic polycycles; mixed aryl and non-aryl polycycles; polyheteroaryl; non-aromatic polyheterocycles; and mixed aryl and non-aryl polyheterocycles;
n, n₁, n₂ and n₃ are the same or different and independently selected from 0-6, when n₁ is 1-6, each carbon atom can be optionally and independently substituted with R₃ and/or R₄;
X and Y are the same or different and independently selected from H; halo; C₁-C₄alkyl, such as CH₃ and CF₃; NO₂; C(O)R₁; OR₉; SR₉; CN; and NR₁₀R₁₁;
R₆ is selected from H; C₁-C₆alkyl; C₄-C₉cycloalkyl; C₄-C₉heterocycloalkyl; cycloalkylalkyl, e.g., cyclopropylmethyl; aryl; heteroaryl; arylalkyl, e.g., benzyl and 2-phenylethenyl; heteroarylalkyl, e.g., pyridylmethyl; OR₁₂; and NR₁₃R₁₄;
R₇ is selected from OR₁₅; SR₁₅; S(O)R₁₆; SO₂R₁₇; NR₁₃R₁₄; and NR₁₂SO₂R₆;
R₈ is selected from H; OR₁₅; NR₁₃R₁₄; C₁-C₆alkyl; C₄-C₉cycloalkyl; C₄-C₉heterocycloalkyl; aryl; heteroaryl; arylalkyl, e.g., benzyl; and heteroarylalkyl, e.g., pyridylmethyl;
R₉ is selected from C₁-C₄alkyl, e.g., CH₃ and CF₃; C(O)-alkyl, e.g., C(O)CH₃; and C(O)CF₃;
R₁₀ and R₁₁ are the same or different and independently selected from H; C₁-C₄alkyl; and —C(O)-alkyl;
R₁₂ is selected from H; C₁-C₆alkyl; C₄-C₉cycloalkyl; C₄-C₉heterocycloalkyl; C₄-C₉heterocycloalkylalkyl; aryl; mixed aryl and non-aryl polycycle; heteroaryl; arylalkyl, e.g., benzyl; and heteroarylalkyl, e.g., pyridylmethyl;
R₁₃ and R₁₄ are the same or different and independently selected from H; C₁-C₆alkyl; C₄-C₉cycloalkyl; C₄-C₉heterocycloalkyl; aryl; heteroaryl; arylalkyl, e.g., benzyl; heteroarylalkyl, e.g., pyridylmethyl; amino acyl; or
R₁₃ and R₁₄, together with the nitrogen to which they are bound, are C₄-C₉heterocycloalkyl; heteroaryl; polyheteroaryl; non-aromatic polyheterocycle; or mixed aryl and non-aryl polyheterocycle;
R₁₅ is selected from H; C₁-C₆alkyl; C₄-C₉cycloalkyl; C₄-C₉heterocycloalkyl; aryl; heteroaryl; arylalkyl; heteroarylalkyl; and (CH₂)ₘZR₁₂;
R₁₆ is selected from C₁-C₆alkyl; C₄-C₉cycloalkyl; C₄-C₉heterocycloalkyl; aryl; heteroaryl; polyheteroaryl; arylalkyl; heteroarylalkyl; and (CH₂)ₘZR₁₂;
R₁₇ is selected from C₁-C₆alkyl; C₄-C₉cycloalkyl; C₄-C₉heterocycloalkyl; aryl; aromatic polycycles; heteroaryl; arylalkyl; heteroarylalkyl; polyheteroaryl and NR₁₃R₁₄;
m is an integer selected from 0-6; and
Z is selected from O; NR₁₃; S; and S(O),
   or a pharmaceutically acceptable salt thereof.

As appropriate, “unsubstituted” means that there is no substituent or that the only substituents are hydrogen.

Halo substituents are selected from fluoro, chloro, bromo and iodo, preferably fluoro or chloro.

Alkyl substituents include straight- and branched-C₁-C₆alkyl, unless otherwise noted. Examples of suitable straight- and branched-C₁-C₆alkyl substituents include methyl, ethyl, n-propyl, 2-propyl, n-butyl, sec-butyl, t-butyl and the like. Unless otherwise noted, the alkyl substituents include both unsubstituted alkyl groups and alkyl groups that are substituted by one or more suitable substituents, including unsaturation, i.e., there are one or more double or triple C—C bonds; acyl; cycloalkyl; halo; oxyalkyl; alkylamino; aminoalkyl; acylamino; and OR₁₅, e.g., alkoxy. Preferred substituents for alkyl groups include halo, hydroxy, alkoxy, oxyalkyl, alkylamino and aminoalkyl.

Cycloalkyl substituents include C₃-C₉cycloalkyl groups, such as cyclopropyl, cyclobutyl, cyclopentyl, cyclohexyl and the like, unless otherwise specified. Unless otherwise noted, cycloalkyl substituents include both unsubstituted cycloalkyl groups and cycloalkyl groups that are substituted by one or more suitable substituents, including C₁-C₆alkyl, halo, hydroxy, aminoalkyl, oxyalkyl, alkylamino and OR₁₅, such as alkoxy. Preferred substituents for cycloalkyl groups include halo, hydroxy, alkoxy, oxyalkyl, alkylamino and aminoalkyl.

The above discussion of alkyl and cycloalkyl substituents also applies to the alkyl portions of other substituents, such as, without limitation, alkoxy, alkyl amines, alkyl ketones, arylalkyl, heteroarylalkyl, alkylsulfonyl and alkyl ester substituents and the like.

Heterocycloalkyl substituents include 3- to 9-membered aliphatic rings, such as 4- to 7-membered aliphatic rings, containing from 1-3 heteroatoms selected from nitrogen, sulfur, oxygen. Examples of suitable heterocycloalkyl substituents include pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane and 1,4-oxathiapane. Unless otherwise noted, the rings are unsubstituted or substituted on the carbon atoms by one or more suitable substituents, including C₁-C₆alkyl; C₄-C₉cycloalkyl; aryl; heteroaryl; arylalkyl, e.g., benzyl; heteroarylalkyl, e.g., pyridylmethyl; halo; amino; alkyl amino and OR₁₅, e.g., alkoxy. Unless otherwise noted, nitrogen heteroatoms are unsubstituted or substituted by H, C₁-C₄alkyl; arylalkyl, e.g., benzyl; heteroarylalkyl, e.g., pyridylmethyl; acyl; aminoacyl; alkylsulfonyl; and arylsulfonyl.

Cycloalkylalkyl substituents include compounds of the formula —(CH₂)ₙ₅-cycloalkyl, wherein n5 is a number from 1-6. Suitable alkylcycloalkyl substituents include cyclopentylmethyl, cyclopentylethyl, cyclohexylmethyl and the like. Such substituents are unsubstituted or substituted in the alkyl portion or in the cycloalkyl portion by a suitable substituent, including those listed above for alkyl and cycloalkyl.

Aryl substituents include unsubstituted phenyl and phenyl substituted by one or more suitable substituents including C₁-C₆alkyl; cycloalkylalkyl, e.g., cyclopropylmethyl; O(CO)alkyl; oxyalkyl; halo; nitro; amino; alkylamino; aminoalkyl; alkyl ketones; nitrile; carboxyalkyl; alkylsulfonyl; aminosulfonyl; arylsulfonyl and OR₁₅, such as alkoxy. Preferred substituents include including C₁-C₆alkyl; cycloalkyl, e.g., cyclopropylmethyl; alkoxy; oxyalkyl; halo; nitro; amino; alkylamino; aminoalkyl; alkyl ketones; nitrile; carboxyalkyl; alkylsulfonyl; arylsulfonyl and aminosulfonyl. Examples of suitable aryl groups include C₁-C₄alkylphenyl, C₁-C₄alkoxyphenyl, trifluoromethylphenyl, methoxyphenyl, hydroxyethylphenyl, dimethylaminophenyl, aminopropylphenyl, carbethoxyphenyl, methanesulfonylphenyl and tolylsulfonylphenyl.

Aromatic polycycles include naphthyl, and naphthyl substituted by one or more suitable substituents including C₁-C₆alkyl; alkylcycloalkyl, e.g., cyclopropylmethyl; oxyalkyl; halo; nitro; amino; alkylamino; aminoalkyl; alkyl ketones; nitrile; carboxyalkyl; alkylsulfonyl; arylsulfonyl; aminosulfonyl and OR₁₅, such as alkoxy.

Heteroaryl substituents include compounds with a 5- to 7-membered aromatic ring containing one or more heteroatoms, e.g., from 1-4 heteroatoms, selected from N, O and S. Typical heteroaryl substituents include furyl, thienyl, pyrrole, pyrazole, triazole, thiazole, oxazole, pyridine, pyrimidine, isoxazolyl, pyrazine and the like. Unless otherwise noted, heteroaryl substituents are unsubstituted or substituted on a carbon atom by one or more suitable substituents, including alkyl, the alkyl substituents identified above, and another heteroaryl substituent. Nitrogen atoms are unsubstituted or substituted, e.g., by R₁₃; especially useful N substituents include H, C₁-C₄alkyl, acyl, aminoacyl and sulfonyl.

Arylalkyl substituents include groups of the formula —(CH₂)ₙ₅-aryl, —(CH₂)ₙ₅₋₁—(CH-aryl)-(CH₂)ₙ₅-aryl or —(CH₂)ₙ₅₋₁CH(aryl)(aryl), wherein aryl and n5 are defined above. Such arylalkyl substituents include benzyl, 2-phenylethyl, 1-phenylethyl, tolyl-3-propyl, 2-phenylpropyl, diphenylmethyl, 2-diphenylethyl, 5,5-dimethyl-3-phenylpentyl and the like. Arylalkyl substituents are unsubstituted or substituted in the alkyl moiety or the aryl moiety or both as described above for alkyl and aryl substituents.

Heteroarylalkyl substituents include groups of the formula —(CH₂)ₙ₅-heteroaryl, wherein heteroaryl and n5 are defined above and the bridging group is linked to a carbon or a nitrogen of the heteroaryl portion, such as 2-, 3- or 4-pyridylmethyl, imidazolylmethyl, quinolylethyl and pyrrolylbutyl. Heteroaryl substituents are unsubstituted or substituted as discussed above for heteroaryl and alkyl substituents.

Amino acyl substituents include groups of the formula —C(O)—(CH₂)ₙ—C(H)(NR₁₃R₁₄)—(CH₂)ₙ—R₅, wherein n, R₁₃, R₁₄ and R₅ are described above. Suitable aminoacyl substituents include natural and non-natural amino acids, such as glycinyl, D-tryptophanyl, L-lysinyl, D- or L-homoserinyl, 4-aminobutryic acyl and ±-3-amin-4-hexenoyl.

Non-aromatic polycycle substituents include bicyclic and tricyclic fused ring systems where each ring can be 4- to 9-membered and each ring can contain zerio, one or more double and/or triple bonds. Suitable examples of non-aromatic polycycles include decalin, octahydroindene, perhydrobenzocycloheptene and perhydrobenzo-[f]-azulene. Such substituents are unsubstituted or substituted as described above for cycloalkyl groups.

Mixed aryl and non-aryl polycycle substituents include bicyclic and tricyclic fused ring systems where each ring can be 4- to 9-membered and at least one ring is aromatic. Suitable examples of mixed aryl and non-aryl polycycles include methylenedioxyphenyl, bis-methylenedioxyphenyl, 1,2,3,4-tetrahydronaphthalene, dibenzosuberane, dihdydroanthracene and 9H-fluorene. Such substituents are unsubstituted or substituted by nitro or as described above for cycloalkyl groups.

Polyheteroaryl substituents include bicyclic and tricyclic fused ring systems where each ring can independently be 5 or 6 membered and contain one or more heteroatom, for example, 1, 2, 3, or 4 heteroatoms, chosen from O, N or S such that the fused ring system is aromatic. Suitable examples of polyheteroaryl ring systems include quinoline, isoquinoline, pyridopyrazine, pyrrolopyridine, furopyridine, indole, benzofuran, benzothiofuran, benzindole, benzoxazole, pyrroloquinoline, and the like. Unless otherwise noted, polyheteroaryl substituents are unsubstituted or substituted on a carbon atom by one or more suitable substituents, including alkyl, the alkyl substituents identified above and a substituent of the formula —O—(CH₂CH═CH(CH₃)(CH₂))₁₋₃H. Nitrogen atoms are unsubstituted or substituted, e.g., by R₁₃, especially useful N substituents include H, C₁-C₄alkyl, acyl, aminoacyl and sulfonyl.

Non-aromatic polyheterocyclic substituents include bicyclic and tricyclic fused ring systems where each ring can be 4- to 9-membered, contain one or more heteroatom, e.g., 1, 2, 3 or 4 heteroatoms, chosen from O, N or S and contain zero or one or more C—C double or triple bonds. Suitable examples of non-aromatic polyheterocycles include hexitol, cis-perhydro-cyclohepta[b]pyridinyl, decahydro-benzo[f][1,4]oxazepinyl, 2,8-dioxabicyclo[3.3.0]octane, hexahydro-thieno[3,2-b]thiophene, perhydropyrrolo[3,2-b]pyrrole, perhydronaphthyridine, perhydro-1H-dicyclopenta[b,e]pyran. Unless otherwise noted, non-aromatic polyheterocyclic substituents are unsubstituted or substituted on a carbon atom by one or more substituents, including alkyl and the alkyl substituents identified above. Nitrogen atoms are unsubstituted or substituted, e.g., by R₁₃, especially useful N substituents include H, C₁-C₄alkyl, acyl, aminoacyl and sulfonyl.

Mixed aryl and non-aryl polyheterocycles substituents include bicyclic and tricyclic fused ring systems where each ring can be 4- to 9-membered, contain one or more heteroatom chosen from O, N or S, and at least one of the rings must be aromatic. Suitable examples of mixed aryl and non-aryl polyheterocycles include 2,3-dihydroindole, 1,2,3,4-tetrahydroquinoline, 5,11-dihydro-10H-dibenz[b,e][1,4]diazepine, 5H-dibenzo[b,e][1,4]diazepine, 1,2-dihydropyrrolo[3,4-b][1,5]benzodiazepine, 1,5-dihydro-pyrido[2,3-b][1,4]diazepin-4-one, 1,2,3,4,6,11-hexahydro-benzo[b]pyrido[2,3-e][1,4]diazepin-5-one. Unless otherwise noted, mixed aryl and non-aryl polyheterocyclic substituents are unsubstituted or substituted on a carbon atom by one or more suitable substituents including —N—OH, ═N—OH, alkyl and the alkyl substituents identified above. Nitrogen atoms are unsubstituted or substituted, e.g., by R₁₃; especially useful N substituents include H, C₁-C₄alkyl, acyl, aminoacyl and sulfonyl.

Amino substituents include primary, secondary and tertiary amines and in salt form, quaternary amines. Examples of amino substituents include mono- and di-alkylamino, mono- and di-aryl amino, mono- and di-arylalkyl amino, aryl-arylalkylamino, alkyl-arylamino, alkyl-arylalkylamino and the like.

Sulfonyl substituents include alkylsulfonyl and arylsulfonyl, e.g., methane sulfonyl, benzene sulfonyl, tosyl and the like.

Acyl substituents include groups of formula —C(O)—W, —OC(O)—W, —C(O)—O—W or —C(O)NR₁₃R₁₄, where W is R₁₆, H or cycloalkylalkyl.

Acylamino substituents include substituents of the formula —N(R₁₂)C(O)—W, —N(R₁₂)C(O)—O—W and —N(R₁₂)C(O)—NHOH and R₁₂ and W are defined above.
The R₂ substituent HON—C(O)—CH═C(R₁)-aryl-alkyl- is a group of the formula

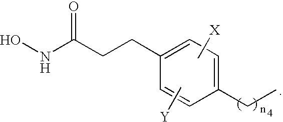

Preferences for each of the substituents include the following:
R₁ is H, halo or a straight-chain C₁-C₄alkyl;
R₂ is selected from H, C₁-C₆alkyl, C₄-C₉cycloalkyl, C₄-C₉heterocycloalkyl, cycloalkylalkyl, aryl, heteroaryl, arylalkyl, heteroarylalkyl, —(CH₂)ₙC(O)R₆, amino acyl and —(CH₂)ₙR₇;
R₃ and R₄ are the same or different and independently selected from H and C₁-C₆alkyl, or
R₃ and R₄, together with the carbon to which they are bound, represent C═O, C═S or C═NR₈;
R₅ is selected from H, C₁-C₆alkyl, C₄-C₉cycloalkyl, C₄-C₉heterocycloalkyl, aryl, heteroaryl, arylalkyl, heteroarylalkyl, a aromatic polycycle, a non-aromatic polycycle, a mixed aryl and non-aryl polycycle, polyheteroaryl, a non-aromatic polyheterocycle, and a mixed aryl and non-aryl polyheterocycle;
n, n₁, n₂ and n₃ are the same or different and independently selected from 0-6, when n₁ is 1-6, each carbon atom is unsubstituted or independently substituted with R₃ and/or R₄;
X and Y are the same or different and independently selected from H, halo, C₁-C₄alkyl, CF₃, NO₂, C(O)R₁, OR₉, SR₉, CN and NR₁₀R₁₁;
R₆ is selected from H, C₁-C₆alkyl, C₄-C₉cycloalkyl, C₄-C₉heterocycloalkyl, alkylcycloalkyl, aryl, heteroaryl, arylalkyl, heteroarylalkyl, OR₁₂ and NR₁₃R₁₄;
R₇ is selected from OR₁₅, SR₁₅, S(O)R₁₆, SO₂R₁₇, NR₁₃R₁₄ and NR₁₂SO₂R₆;
R₈ is selected from H, OR₁₅, NR₁₃R₁₄, C₁-C₆alkyl, C₄-C₉cycloalkyl, C₄-C₉heterocycloalkyl, aryl, heteroaryl, arylalkyl and heteroarylalkyl;
R₉ is selected from C₁-C₄alkyl and C(O)-alkyl;
R₁₀ and R₁₁ are the same or different and independently selected from H, C₁-C₄alkyl and —C(O)-alkyl;
R₁₂ is selected from H, C₁-C₆alkyl, C₄-C₉cycloalkyl, C₄-C₉heterocycloalkyl, aryl, heteroaryl, arylalkyl and heteroarylalkyl;
R₁₃ and R₁₄ are the same or different and independently selected from H, C₁-C₆alkyl, C₄-C₉cycloalkyl, C₄-C₉heterocycloalkyl, aryl, heteroaryl, arylalkyl, heteroarylalkyl and amino acyl;
R₁₅ is selected from H, C₁-C₆alkyl, C₄-C₉cycloalkyl, C₄-C₉heterocycloalkyl, aryl, heteroaryl, arylalkyl, heteroarylalkyl and (CH₂)ₘZR₁₂;
R₁₆ is selected from C₁-C₆alkyl, C₄-C₉cycloalkyl, C₄-C₉heterocycloalkyl, aryl, heteroaryl, arylalkyl, heteroarylalkyl and (CH₂)ₘZR₁₂;
R₁₇ is selected from C₁-C₆alkyl, C₄-C₉cycloalkyl, C₄-C₉heterocycloalkyl, aryl, heteroaryl, arylalkyl, heteroarylalkyl and NR₁₃R₁₄;
m is an integer selected from 0-6; and
Z is selected from O, NR₁₃, S and S(O);
   or a pharmaceutically acceptable salt thereof.

Useful compounds of the formula (I), include those wherein each of R₁, X, Y, R₃ and R₄ is H, including those wherein one of n₂ and n₃ is 0 and the other is 1, especially those wherein R₂ is H or —CH₂—CH₂—OH.

One suitable genus of hydroxamate compounds are those of formula (IIIa)

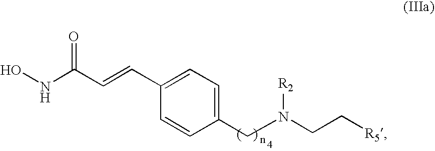

wherein
n₄ is 0-3;
R₂ is selected from H, C₁-C₆alkyl, C₄-C₉cycloalkyl, C₄-C₉heterocycloalkyl, cycloalkylalkyl, aryl, heteroaryl, arylalkyl, heteroarylalkyl, —(CH₂)ₙC(O)R₆, amino acyl and —(CH₂)ₙR₇; and
R₅ is heteroaryl; heteroarylalkyl, e.g., pyridylmethyl; aromatic polycycles; non-aromatic polycycles; mixed aryl and non-aryl polycycles; polyheteroaryl or mixed aryl; and non-aryl polyheterocycles;
   or a pharmaceutically acceptable salt thereof.

Another suitable genus of hydroxamate compounds are those of formula (IIIa)

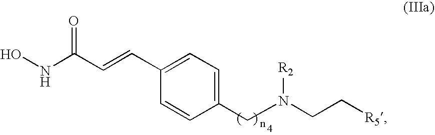

wherein
n₄ is 0-3;
R₂ is selected from H, C₁-C₆alkyl, C₄-C₉cycloalkyl, C₄-C₉heterocycloalkyl, cycloalkylalkyl, aryl, heteroaryl, arylalkyl, heteroarylalkyl, —(CH₂)ₙC(O)R₆, amino acyl and —(CH₂)ₙR₇;
R₅′ is aryl; arylalkyl; aromatic polycycles; non-aromatic polycycles and mixed aryl; and non-aryl polycycles, especially aryl, such as p-fluorophenyl, p-chlorophenyl, p-O—C₁-C₄alkylphenyl, such as p-methoxyphenyl, and p-C₁-C₄alkylphenyl; and arylalkyl, such as benzyl, ortho-, meta- or para-fluorobenzyl, ortho-, meta- or para-chlorobenzyl, ortho-, meta- or para-mono, di- or tri-O—C₁-C₄alkylbenzyl, such as ortho-, meta- or para-methoxybenzyl, m,p-diethoxybenzyl, o,m,p-triimethoxybenzyl and ortho-, meta- or para-mono, di- or tri-C₁-C₄alkylphenyl, such as p-methyl, m,m-diethylphenyl;
   or a pharmaceutically acceptable salt thereof.

Another interesting genus is the compounds of formula (IIIb)

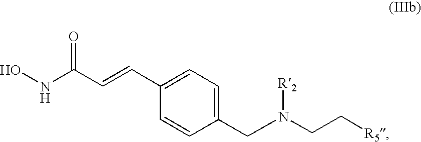

wherein
R₂′ is selected from H; C₁-C₆alkyl; C₄-C₆cycloalkyl; cycloalkylalkyl, e.g., cyclopropylmethyl; (CH₂)₂₋₄OR₂₁, where R₂₁ is H, methyl, ethyl, propyl and i-propyl; and
R₅″ is unsubstituted 1H-indol-3-yl, benzofuran-3-yl or quinolin-3-yl, or substituted 1H-indol-3-yl, such as 5-fluoro-1H-indol-3-yl or 5-methoxy-1H-indol-3-yl, benzofuran-3-yl or quinolin-3-yl;
   or a pharmaceutically acceptable salt thereof.

Another interesting genus of hydroxamate compounds are the compounds of formula (IIIc)

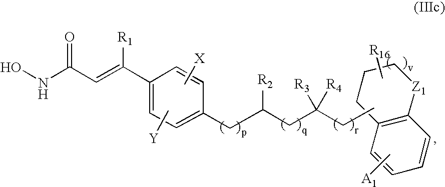

wherein
the ring containing Z₁ is aromatic or non-aromatic, which non-aromatic rings are saturated or unsaturated,
Z₁ is O, S or N—R₂₀;
R₁₈ is H; halo; C₁-C₆alkyl (methyl, ethyl, t-butyl); C₃-C₇cycloalkyl; aryl, e.g., unsubstituted phenyl or phenyl substituted by 4-OCH₃ or 4-CF₃; or heteroaryl, such as 2-furanyl, 2-thiophenyl or 2-, 3- or 4-pyridyl;
R₂₀ is H; C₁-C₆alkyl; C₁-C₆alkyl-C₃-C₉cycloalkyl, e.g., cyclopropylmethyl; aryl; heteroaryl; arylalkyl, e.g., benzyl; heteroarylalkyl, e.g., pyridylmethyl; acyl, e.g., acetyl, propionyl and benzoyl; or sulfonyl, e.g., methanesulfonyl, ethanesulfonyl, benzenesulfonyl and toluenesulfonyl;
A₁ is 1, 2 or 3 substituents which are independently H; C₁-C₆alkyl; —OR₁₉; halo; alkylamino; aminoalkyl; halo; or heteroarylalkyl, e.g., pyridylmethyl;
   R₁₉ is selected from H; C₁-C₆alkyl; C₄-C₉cycloalkyl; C₄-C₉heterocycloalkyl; aryl; heteroaryl; arylalkyl, e.g., benzyl; heteroarylalkyl, e.g., pyridylmethyl and —(CH₂CH═CH(CH₃)(CH₂))₁₋₃H;
R₂ is selected from H, C₁-C₆alkyl, C₄-C₉cycloalkyl, C₄-C₉heterocycloalkyl, cycloalkylalkyl, aryl, heteroaryl, arylalkyl, heteroarylalkyl, —(CH₂)ₙC(O)R₆, amino acyl and —(CH₂)ₙR₇;
v is 0, 1 or 2;
p is 0-3; and
q is 1-5 and r is 0; or
q is 0 and r is 1-5;
   or a pharmaceutically acceptable salt thereof. The other variable substituents are as defined above.

Especially useful compounds of formula (IIIc), are those wherein R₂ is H, or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3, especially those wherein Z₁ is N—R₂₀. Among these compounds R₂ is preferably H or —CH₂—CH₂—OH and the sum of q and r is preferably 1.

Another interesting genus of hydroxamate compounds are the compounds of formula (IIId)

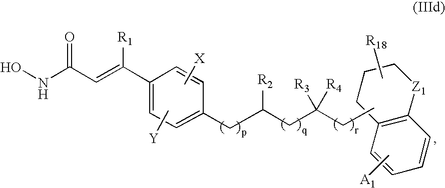

wherein
Z₁ is O, S or N—R₂₀;
R₁₈ is H; halo; C₁-C₆alkyl (methyl, ethyl, t-butyl); C₃-C₇cycloalkyl; aryl, e.g., unsubstituted phenyl or phenyl substituted by 4-OCH₃ or 4-CF₃; or heteroaryl;
   R₂₀ is H; C₁-C₆alkyl, C₁-C₆alkyl-C₃-C₉cycloalkyl, e.g., cyclopropylmethyl; aryl; heteroaryl; arylalkyl, e.g., benzyl; heteroarylalkyl, e.g., pyridylmethyl; acyl, e.g., acetyl, propionyl and benzoyl; or sulfonyl, e.g., methanesulfonyl, ethanesulfonyl, benzenesulfonyl, toluenesulfonyl);
A₁ is 1, 2 or 3 substituents which are independently H, C₁-C₆alkyl, —OR₁₉ or halo;
   R₁₉ is selected from H; C₁-C₆alkyl; C₄-C₉cycloalkyl; C₄-C₉heterocycloalkyl; aryl; heteroaryl; arylalkyl, e.g., benzyl; and heteroarylalkyl, e.g., pyridylmethyl;
p is 0-3; and
q is 1-5 and r is 0; or
q is 0 and r is 1-5;
   or a pharmaceutically acceptable salt thereof. The other variable substituents are as defined above.

Especially useful compounds of formula (IIId), are those wherein R₂ is H or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

The present invention further relates to compounds of the formula (IIIe)

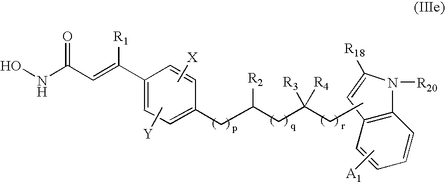

or a pharmaceutically acceptable salt thereof. The variable substituents are as defined above.

Especially useful compounds of formula (IIIe), are those wherein R₁₈ is H, fluoro, chloro, bromo, a C₁-C₄alkyl group, a substituted C₁-C₄alkyl group, a C₃-C₇cycloalkyl group, unsubstituted phenyl, phenyl substituted in the para position, or a heteroaryl, e.g., pyridyl, ring.

Another group of useful compounds of formula (IIIe), are those wherein R₂ is H or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1. Among these compounds p is preferably 1 and R₃ and R₄ are preferably H.

Another group of useful compounds of formula (IIIe), are those wherein R₁₈ is H, methyl, ethyl, t-butyl, trifluoromethyl, cyclohexyl, phenyl, 4-methoxyphenyl, 4-trifluoromethylphenyl, 2-furanyl, 2-thiophenyl, or 2-, 3- or 4-pyridyl wherein the 2-furanyl, 2-thiophenyl and 2-, 3- or 4-pyridyl substituents are unsubstituted or substituted as described above for heteroaryl rings; R₂ is H or —(CH₂)ₚCH₂OH, wherein p is 1-3; especially those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

Those compounds of formula (IIIe), wherein R₂₀ is H or C₁-C₆alkyl, especially H, are important members of each of the subgenuses of compounds of formula (Ie) described above.

N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(1H-indol-3-yl)ethyl]-amino]methyl]phenyl]-2E-2-propenamide and N-hydroxy-3-[4-[[[2-(2-methyl-1H-indol-3-yl)-ethyl]-amino]methyl]phenyl]-2E-2-propenamide or a pharmaceutically acceptable salt thereof, are important compounds of formula (IIIe).

The present invention further relates to the compounds of the formula (IIIf)

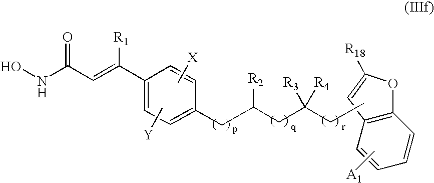

or a pharmaceutically acceptable salt thereof. The variable substituents are as defined above.

Useful compounds of formula (IIIf), are include those wherein R₂ is H or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

N-hydroxy-3-[4-[[[2-(benzofur-3-yl)-ethyl]-amino]methyl]phenyl]-2E-2-propenamide or a pharmaceutically acceptable salt thereof, is an important compound of formula (IIIf).

The compounds described above are often used in the form of a pharmaceutically acceptable salt. Pharmaceutically acceptable salts include, when appropriate, pharmaceutically acceptable base addition salts and acid addition salts, for example, metal salts, such as alkali and alkaline earth metal salts, ammonium salts, organic amine addition salts and amino acid addition salts and sulfonate salts. Acid addition salts include inorganic acid addition salts, such as hydrochloride, sulfate and phosphate; and organic acid addition salts, such as alkyl sulfonate, arylsulfonate, acetate, maleate, fumarate, tartrate, citrate and lactate. Examples of metal salts are alkali metal salts, such as lithium salt, sodium salt and potassium salt; alkaline earth metal salts, such as magnesium salt and calcium salt, aluminum salt and zinc salt. Examples of ammonium salts are ammonium salt and tetramethylammonium salt. Examples of organic amine addition salts are salts with morpholine and piperidine. Examples of amino acid addition salts are salts with glycine, phenylalanine, glutamic acid and lysine. Sulfonate salts include mesylate, tosylate and benzene sulfonic acid salts.

Additional HDAI compounds within the scope of formula (I), and their synthesis, are disclosed in WO 02/22577 published Mar. 21, 2002 which is incorporated herein by reference in its entirety. Two preferred compounds within the scope of WO 02/22577 are

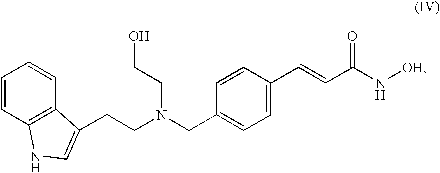

N-hydroxy-3-[4-[(2-hydroxyethyl){2-(1H-indol-3-yl)ethyl]-amino]methyl]phenyl]-2E-2-propenamide, or a pharmaceutically acceptable salt thereof hereinafter “Compound (IV)” and

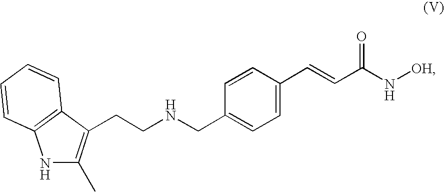

N-hydroxy-3-[4-[[[2-(2-methyl-1H-indol-3-yl)-ethyl]-amino]methyl]phenyl]-2E-2-propenamide, or a pharmaceutically acceptable salt thereof, hereinafter “Compound (V)”.

In other embodiments of the present invention the HDAC inhibitor compound may be selected from any compound that inhibits histone deacetylase such as compounds selected from trapoxin and other tetrapeptides e.g. chlamydocin and HC Toxin; trichostatin and its analogues such as trichostatin A; apicidin; suberoylanilide hydroxamic acid (SAHA); oxamflatin; MS-275; pyroxamide; valproic acid; [4-(2-amino-phenylcarbamoyl)-benzyl]-carbamic acid pyridine-3-ylmethyl ester and derivatives thereof; Depsipeptide FR901228; CI-994; phenylbutyrate; sodium butyrate; butyric acid; 3-(4-aroyl-1H-2pyrrolyl-N-hydroxy-propenamides as disclosed in J. Med. Chem. 45(9):1778-84 (Apr. 25, 2002); ADHA compound 8; -(−)Depudecin; Scriptaid; and Sirtinol.

In each case where citations of patent applications are given above, the subject matter relating to the compounds is hereby incorporated into the present application by reference. Comprised are likewise the pharmaceutically acceptable salts thereof, the corresponding racemates, diastereoisomers, enantiomers, tautomers, as well as the corresponding crystal modifications of above disclosed compounds where present, e.g., solvates, hydrates and polymorphs, which are disclosed therein. The compounds used as active ingredients in the combinations of the invention can be prepared and administered as described in the cited documents, respectively. Also within the scope of this invention is the combination of more than two separate active ingredients as set forth above, i.e., a pharmaceutical combination within the scope of this invention could include three active ingredients or more.

In accordance with the particular findings of the present invention, there is provided
1. A pharmaceutical combination comprising:
   a) a pyrimidylaminobenzamide compound of formula (I); and
   b) at least one HDAC inhibitor.
2. A method for treating or preventing proliferative disease in a subject in need thereof, comprising co-administration to said subject, e.g., concomitantly or in sequence, of a therapeutically effective amount of a pyrimidylaminobenzamide compound of formula (I) and an HDAC inhibitor, e.g., as disclosed above.
Examples of proliferative diseases include e.g. tumors, leukemias, psoriasis, restenosis, sclerodermitis and fibrosis.
3. A pharmaceutical combination as defined under 1) above, e.g., for use in a method as defined under 2) above.
4. A pharmaceutical combination as defined under 1) above for use in the preparation of a medicament for use in a method as defined under 2) above.

Utility of the combination of the invention in a method as hereinabove specified, may be demonstrated in animal test methods as well as in clinic, for example, in accordance with the methods hereinafter described.

It has now surprisingly been found that the combination of a pyrimidylaminobenzamide compound and an HDAC inhibitor possesses therapeutic properties, which render it particularly useful as a treatment for proliferative diseases.

In another embodiment, the instant invention provides a method for treating proliferative diseases comprising administering to a mammal in need of such treatment a therapeutically effective amount of the combination of a pyrimidylaminobenzamide compound and an HDAC inhibitor or pharmaceutically acceptable salts or prodrugs thereof.

Preferably the instant invention provides a method for treating mammals, especially humans, suffering from proliferative diseases comprising administering to a mammal in need of such treatment an inhibiting amount of the combination of a pyrimidylaminobenzamide compound and an HDAC inhibitor or pharmaceutically acceptable salts thereof.

In the present description, the term “treatment” includes both prophylactic or preventative treatment as well as curative or disease suppressive treatment, including treatment of patients at risk of contracting the disease or suspected to have contracted the disease as well as ill patients. This term further includes the treatment for the delay of progression of the disease.

The term “curative”, as used herein, means efficacy in treating ongoing episodes involving proliferative diseases.

The term “prophylactic” means the prevention of the onset or recurrence of diseases involving proliferative diseases.

The term “delay of progression”, as used herein, means administration of the active compound to patients being in a pre-stage or in an early phase of the disease to be treated, in which patients, for example, a pre-form of the corresponding disease is diagnosed or which patients are in a condition, e.g., during a medical treatment or a condition resulting from an accident, under which it is likely that a corresponding disease will develop.

This unforeseeable range of properties means that the use of the combination of a pyrimidylaminobenzamide compound and an HDAC inhibitor are of particular interest for the manufacture of a medicament for the treatment of proliferative diseases.

To demonstrate that the combination of a pyrimidylaminobenzamide compound and an HDAC inhibitor is particularly suitable for the treatment of proliferative diseases with good therapeutic margin and other advantages, clinical trials can be carried out in a manner known to the skilled person.

### A. Combined Treatment

Suitable clinical studies are, for example, open label, dose escalation studies in patients with proliferative diseases. Such studies prove in particular the synergism of the active ingredients of the combination of the invention. The beneficial effects can be determined directly through the results of these studies which are known as such to a person skilled in the art. Such studies are, in particular, suitable to compare the effects of a monotherapy using the active ingredients and a combination of the invention. Preferably, the dose of agent (a) is escalated until the Maximum Tolerated Dosage is reached, and agent (b) is administered with a fixed dose. Alternatively, the agent (a) is administered in a fixed dose and the dose of agent (b) is escalated. Each patient receives doses of the agent (a) either daily or intermittent. The efficacy of the treatment can be determined in such studies, e.g., after 12, 18 or 24 weeks by evaluation of symptom scores every 6 weeks.

The administration of a pharmaceutical combination of the invention results not only in a beneficial effect, e.g., a synergistic therapeutic effect, e.g., with regard to alleviating, delaying progression of or inhibiting the symptoms, but also in further surprising beneficial effects, e.g., fewer side effects, an improved quality of life or a decreased morbidity, compared with a monotherapy applying only one of the pharmaceutically active ingredients used in the combination of the invention.

A further benefit is that lower doses of the active ingredients of the combination of the invention can be used, for example, that the dosages need not only often be smaller but are also applied less frequently, which may diminish the incidence or severity of side-effects. This is in accordance with the desires and requirements of the patients to be treated.

The term “co-administration” or “combined administration” or the like as utilized herein are meant to encompass administration of the selected therapeutic agents to a single patient, and are intended to include treatment regimens in which the agents are not necessarily administered by the same route of administration or at the same time.

It is one objective of this invention to provide a pharmaceutical composition comprising a quantity, which is jointly therapeutically effective at targeting or preventing proliferative diseases a combination of the invention. In this composition, agent (a) and agent (b) may be administered together, one after the other or separately in one combined unit dosage form or in two separate unit dosage forms. The unit dosage form may also be a fixed combination.

The pharmaceutical compositions for separate administration of agent (a) and agent (b) or for the administration in a fixed combination, i.e., a single galenical composition comprising at least two combination partners (a) and (b), according to the invention may be prepared in a manner known per se and are those suitable for enteral, such as oral or rectal, and parenteral administration to mammals (warm-blooded animals), including humans, comprising a therapeutically effective amount of at least one pharmacologically active combination partner alone, e.g., as indicated above, or in combination with one or more pharmaceutically acceptable carriers or diluents, especially suitable for enteral or parenteral application.

Suitable pharmaceutical compositions contain, for example, from about 0.1% to about 99.9%, preferably from about 1% to about 60%, of the active ingredient(s). Pharmaceutical preparations for the combination therapy for enteral or parenteral administration are, for example, those in unit dosage forms, such as sugar-coated tablets, tablets, capsules or suppositories, or ampoules. If not indicated otherwise, these are prepared in a manner known per se, for example, by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

In particular, a therapeutically effective amount of each of the combination partner of the combination of the invention may be administered simultaneously or sequentially and in any order, and the components may be administered separately or as a fixed combination. For example, the method of preventing or treating proliferative diseases according to the invention may comprise: (i) administration of the first agent (a) in free or pharmaceutically acceptable salt form; and (ii) administration of an agent (b) in free or pharmaceutically acceptable salt form, simultaneously or sequentially in any order, in jointly therapeutically effective amounts, preferably in synergistically effective amounts, e.g., in daily or intermittently dosages corresponding to the amounts described herein. The individual combination partners of the combination of the invention may be administered separately at different times during the course of therapy or concurrently in divided or single combination forms. Furthermore, the term administering also encompasses the use of a pro-drug of a combination partner that convert in vivo to the combination partner as such. The instant invention is therefore, to be understood as embracing all such regimens of simultaneous or alternating treatment and the term “administering” is to be interpreted accordingly.

The effective dosage of each of the combination partners employed in the combination of the invention may vary depending on the particular compound or pharmaceutical composition employed, the mode of administration, the condition being treated, the severity of the condition being treated. Thus, the dosage regimen of the combination of the invention is selected in accordance with a variety of factors including the route of administration and the renal and hepatic function of the patient. A clinician or physician of ordinary skill can readily determine and prescribe the effective amount of the single active ingredients required to alleviate, counter or arrest the progress of the condition. Optimal precision in achieving concentration of the active ingredients within the range that yields efficacy without toxicity requires a regimen based on the kinetics of the active ingredients' availability to target sites.

Daily dosages for agent (a) or (b) or will, of course, vary depending on a variety of factors, for example, the compound chosen, the particular condition to be treated and the desired effect. In general, however, satisfactory results are achieved on administration of agent (a) at daily dosage rates of the order of ca. 0.03-5 mg/kg per day, particularly 0.1-5 mg/kg per day, e.g., 0.1-2.5 mg/kg per day, as a single dose or in divided doses. Agent (a) and agent (b) may be administered by any conventional route, in particular, enterally, e.g., orally, e.g., in the form of tablets, capsules, drink solutions or parenterally, e.g., in the form of injectable solutions or suspensions. Suitable unit dosage forms for oral administration comprise from ca. 0.02-50 mg active ingredient, usually 0.1-30 mg, e.g., agent (a) or (b), together with one or more pharmaceutically acceptable diluents or carriers therefore.

Agent (b) may be administered to a human in a daily dosage range of 0.5-1,000 mg. Suitable unit dosage forms for oral administration comprise from ca. 0.1-500 mg active ingredient, together with one or more pharmaceutically acceptable diluents or carriers therefore.

The administration of a pharmaceutical combination of the invention results not only in a beneficial effect, e.g., a synergistic therapeutic effect, e.g., with regard to inhibiting the unregulated proliferation of haematological stem cells or slowing down the progression of leukemias, such as chronic myeloid leukemia (CML), acute lymphocyte leukemia (ALL) or acute myeloid leukemia (AML), or the growth of tumors, but also in further surprising beneficial effects, e.g., less side effects, an improved quality of life or a decreased morbidity, compared to a monotherapy applying only one of the pharmaceutically active ingredients used in the combination of the invention.

A further benefit is that lower doses of the active ingredients of the combination of the invention can be used, for example, that the dosages need not only often be smaller but are also applied less frequently, or can be used in order to diminish the incidence of side-effects. This is in accordance with the desires and requirements of the patients to be treated.

Combinations of a pyrimidylaminobenzamide compound and an HDAC inhibitor may be combined, independently or together, with one or more pharmaceutically acceptable carriers and, optionally, one or more other conventional pharmaceutical adjuvants and administered enterally, e.g., orally, in the form of tablets, capsules, caplets, etc. or parenterally, e.g., intraperitoneally or intravenously, in the form of sterile injectable solutions or suspensions. The enteral and parenteral compositions may be prepared by conventional means.

The combination of a pyrimidylaminobenzamide compound and an HDAC inhibitor can be used alone or combined with at least one other pharmaceutically active compound for use in these pathologies. These active compounds can be combined in the same pharmaceutical preparation or in the form of combined preparations “kit of parts” in the sense that the combination partners can be dosed independently or by use of different fixed combinations with distinguished amounts of the combination partners, i.e., simultaneously or at different time points. The parts of the kit of parts can then, e.g., be administered simultaneously or chronologically staggered, that is at different time points and with equal or different time intervals for any part of the kit of parts. Non-limiting examples of compounds which can be cited for use in combination with the combination of a pyrimidylaminobenzamide compound and an HDAC inhibitor are cytotoxic chemotherapy drugs, such as cytosine arabinoside, daunorubicin, doxorubicin, cyclophosphamide, VP-16, or imatinib etc. Further, the combination of a pyrimidylaminobenzamide compound and an HDAC inhibitor could be combined with other inhibitors of signal transduction or other oncogene-targeted drugs with the expectation that significant synergy would result.

### B. Diseases to be Treated

The term “proliferative disease” includes but is not restricted to tumors, psoriasis, restenosis, sclerodermitis and fibrosis.

The term hematological malignancy, refers in particular to leukemias, especially those expressing Bcr-Abl, c-Kit or HDAC, (or those depending on Bcr-Abl, c-Kit or HDAC) and includes, but is not limited to, CML and ALL, especially the Philadelphia chromosome positive ALL (Ph+ALL), as well as Imatinib-resistant leukemia. Especially preferred is use of the combinations of the present invention for leukemias, such as CML, ALL or AML. Most especially preferred is use in diseases which show resistance to Imatinib. (Imatinib and is sold under the name Gleevec®).

The term “a solid tumor disease” especially means ovarian cancer, breast cancer, cancer of the colon and generally the gastrointestinal tract, cervix cancer, lung cancer, e.g., small-cell lung cancer and non-small-cell lung cancer, head and neck cancer, bladder cancer, cancer of the prostate or Kaposi's sarcoma.

The combinations according to the invention, that inhibit the protein kinase activities mentioned, especially tyrosine protein kinases mentioned above and below, can therefore be used in the treatment of protein kinase dependent diseases. Protein kinase dependent diseases are especially proliferative diseases, preferably benign or especially malignant tumours (for example, carcinoma of the kidneys, brain, liver, adrenal glands, bladder, breast, stomach (especially gastric tumors), ovaries, colon, rectum, prostate, pancreas, lungs (especially SCLC), vagina or thyroid, sarcoma, multiple myeloma, glioblastomas and numerous tumours of the neck and head, as well as leukemias); especially colon carcinoma or colorectal adenoma, or a tumor of the neck and head, an epidermal hyperproliferation, especially psoriasis, prostate hyperplasia, a neoplasia, especially of epithelial character, preferably mammary carcinoma, or a leukemia. They are able to bring about the regression of tumours and to prevent the formation of tumor metastases and the growth of (also micro) metastases. In addition they can be used in epidermal hyperproliferation (e.g., psoriasis), in prostate hyperplasia, and in the treatment of neoplasias, especially of epithelial character, for example, mammary carcinoma. It is also possible to use the combinations of the present invention in the treatment of diseases of the immune system insofar as several or, especially, individual tyrosine protein kinases are involved; furthermore, the combinations of the present invention can be used also in the treatment of diseases of the central or peripheral nervous system where signal transmission by at least one tyrosine protein kinase, especially selected from those mentioned specifically, is involved.

In CML, a reciprocally balanced chromosomal translocation in hematopoietic stem cells (HSCs) produces the Bcr-Abl hybrid gene. The latter encodes the oncogenic Bcr-Abl fusion protein. Whereas Abl encodes a tightly regulated protein tyrosine kinase, which plays a fundamental role in regulating cell proliferation, adherence and apoptosis, the Bcr-Abl fusion gene encodes as constitutively activated kinase, which transforms HSCs to produce a phenotype exhibiting deregulated clonal proliferation, reduced capacity to adhere to the bone marrow stroma and a reduces apoptotic response to mutagenic stimuli, which enable it to accumulate progressively more malignant transformations. The resulting granulocytes fail to develop into mature lymphocytes and are released into the circulation, leading to a deficiency in the mature cells and increased susceptibility to infection. ATP-competitive inhibitors of Bcr-Abl have been described which prevent the kinase from activating mitogenic and anti-apoptotic pathways (e.g. P-3 kinase and STAT5), leading to the death of the Bcr-Abl phenotype cells and thereby providing an effective therapy against CML. The combinations of the present invention are thus especially appropriate for the therapy of diseases related to its overexpression, especially leukemias, such as leukemias, e.g., CML or ALL.

In a broader sense of the invention, a proliferative disease includes hyperproliferative conditions, such as leukemias, hyperplasias, fibrosis (especially pulmonary, but also other types of fibrosis, such as renal fibrosis), angiogenesis, psoriasis, atherosclerosis and smooth muscle proliferation in the blood vessels, such as stenosis or restenosis following angioplasty. In another aspect the combinations of the present invention could be used to treat arthritis.

Combinations of the present invention can also be used to treat or prevent fibrogenic disorders such as scleroderma (systemic sclerosis); diseases associated with protein aggregation and amyloid formation, such as Huntington's disease; inhibition of the replication of hepatitis C virus and treating hepatitis C virus; treating tumors associated with viral infection, such as human papilloma virus; and inhibiting viruses dependent of heat-shock proteins.

The combinations of the present invention primarily inhibit the growth of blood vessels and are thus, for example, effective against a number of diseases associated with deregulated angiogenesis, especially diseases caused by ocular neovascularisation, especially retinopathies, such as diabetic retinopathy or age-related macula degeneration, psoriasis, hemangioblastoma, such as hemangioma, mesangial cell proliferative disorders, such as chronic or acute, renal diseases, e.g., diabetic nephropathy, malignant nephrosclerosis, thrombotic microangiopathy syndromes or transplant rejection, or especially inflammatory renal disease, such as glomerulonephritis, especially mesangioproliferative glomerulonephritis, hemolytic-uraemic syndrome, diabetic nephropathy, hypertensive nephrosclerosis, atheroma, arterial restenosis, autoimmune diseases, diabetes, endometriosis, chronic asthma, and especially neoplastic diseases (solid tumors, but also leukemias and other haematological malignancies), such as especially breast cancer, cancer of the colon, lung cancer (especially small-cell lung cancer), cancer of the prostate or Kaposi's sarcoma. Combinations of the present invention inhibit the growth of tumors and are especially suited to preventing the metastatic spread of tumors and the growth of micrometastases.

Combinations of the present invention may in particular be used to treat:
(i) a breast tumor; an epidermoid tumor, such as an epidermoid head and/or neck tumor or a mouth tumor; a lung tumor, e.g., a small cell or non-small cell lung tumor; a gastrointestinal tumor, e.g., a colorectal tumor; or a genitourinary tumor, e.g., a prostate tumor, especially a hormone-refractory prostate tumor;
(ii) a proliferative disease that is refractory to the treatment with other chemotherapeutics; or
(iii) a tumor that is refractory to treatment with other chemotherapeutics due to multidrug resistance.

### EXAMPLE

Reagents and antibodies: Compound (V) and Compound (II) are provided by Novartis Pharmaceuticals (East Hanover, N.J.). Polyclonal anti-PARP, anti-caspase-9, anti-caspase-3, and anti-p-ERK1/2 antibodies are purchased from Cell Signaling Technology (Beverly, Mass.). Polyclonal anti-STAT-5 and goat polyclonal anti-Pim-2 antibody, as well as monoclonal anti-c-Myc and anti-Abl antibodies, are purchased from Santa Cruz Biotechnology (Santa Cruz, Calif.). Monoclonal anti-p-STAT5 antibody is purchased from Upstate Biotechnology (Lake Placid, N.Y.). Antibodies for the immunoblot analyses of p21, p27, p-CrkL, CrkL, p-AKT, AKT, Bim, Bcl-x_{L} and ERK1/2 are obtained as previously described.

Cell lines and cell culture: Bcr-Abl-expressing, CML LAMA-84 and K562 cells are obtained from American Tissue Culture Collection (Manassas, Va.) and maintained in culture in RPMI medium containing 10% fetal bovine serum and passaged twice a week. Mouse pro-B BaF3 cells are cultured in complete RPMI-1640 media supplemented with 10% WEHI medium as the source of IL-3. For the studies described herein, logarithmically growing cells are exposed to the designated concentrations of Compound (II) and/or Compound (V). Following these treatments, cells or cell pellets are washed free of the drug(s) prior to the performance of the studies.

Site-directed mutagenesis and nucleofection: Three p210 Bcr-Abl constructs are used in the current studies. The p210 Bcr-Abl WT and p210 Bcr-Abl (T315I) constructs are generated. The p210 Bcr-Abl (E255K) mutant is created by site-directed mutagenesis of a Bcr-Abl containing pSVneo construct using a QuikChange II XL kit (Stratagene, Cedar Creek, Tex.) according to the manufacturer's recommendations, and the resulting clones are sequenced to confirm the point mutation. For nucleofection of the p210 Bcr-Abl constructs into BaF3 cells, 5 million BaF3 cells in 100 μL Nucleofector solution V (Amaxa, Gaithersburg, Md.) are mixed with 5 μg p210 Bcr-Abl WT, p210 Bcr-Abl (T315I), or p210 Bcr-Abl (E255K) in a cuvette and nucleofected using program G-16. Following nucleofection, the cells are incubated at a concentration of 1×10⁶ cells/mL in complete RPMI-1640 media supplemented with 10% WEHI medium as the source of IL-3, overnight, to recover. Stable transfectants of BaF3 cells expressing the WT or mutant form of Bcr-Abl (i.e., T315I or E255K) are maintained in RPMI 1640 supplemented with 10% serum, 1.0 U/mL penicillin, 1 μg/mL streptomycin and 0.75 mg/mL G418. Stably expressing cells are then further selected by removal of IL-3. After confirmation of Bcr-Abl expression by immunoblot analysis, cells are used for the studies described herein.

Primary CML-BC cells and NBMCs: Leukemia cells from the peripheral blood and/or bone marrow of 10 patients who had met the clinical criteria of imatinib-resistant Ph chromosome-positive CML-BC (Imatinib-resistant Philadelphia chromosome-positive chromic myeloid leukaemia-blast crisis) are harvested and purified. Additionally, NBMCs are harvested and purified. Informed consents are signed by all patients to allow use of their cells for these experiments, as part of a clinical protocol approved by the University of South Florida Institutional Review Board (IRB).

Sequencing of Bcr-Abl in CML-BC cells: Using the Trizol method (Invitrogen, Carlsbad, Calif.), total RNA is isolated from 10-15 million cells available from 2 patients, who are suspected to have Bcr-Abl T315I mutation as a result of their failure to respond to treatment with imatinib and Compound (II). Total RNA (5 μg) is reverse transcribed with a first-strand cDNA synthesis kit (Invitrogen). Reverse transcribed cDNAs are used in polymerase chain reaction (PCR) amplifications to amplify a fragment of Bcr-Abl that included the Bcr junction region and the c-Abl kinase region. The amplified sequences are agarose gel-purified and cloned into pCR4-TOPO plasmid. The resulting plasmids are transformed into Escherichia coli Mach1 cells (Invitrogen) overnight at 37° C. Ten colonies for each sample are checked by colony PCR and subcultured for plasmid isolation. Isolated plasmids are sequence verified with T3 and T7 primers for c-Abl kinase domain mutations.

Suspension culture or colony growth inhibition: Following treatment with the designated concentrations of Compound (II) and/or Compound (V) for 48 hours, untreated and drug-treated cells are washed in RPMI 1640 medium. Following this, cells are placed in suspension culture at a concentration of 200000 cells/mL for 4 days. At the end of this incubation period, cell concentrations and percentage increase in cell numbers are determined. Alternatively, following treatment with the drugs, approximately 200 cells treated under each condition are resuspended in 100 μL RPMI 1640 media containing 10% FBS, then plated in duplicate wells in a 12-well plate containing 1.0 mL Methocult media (Stem Cell Technologies, Vancouver, Canada) per well, according to the manufacturer's protocol. The plates are placed in an incubator at 37° C. with 5% CO₂ for 10 days. Following this incubation, colonies consisting of 50 or more cells, in each well, are counted by an inverted microscope, and the percentage of colony growth inhibition compared with the untreated control cells is calculated.

Assessment of percentage of non-viable cells: Cells are stained with trypan blue (Sigma, St Louis, Mo.). The numbers of non-viable cells are determined by counting the cells that showed trypan blue uptake in a hemocytometer and reported as the percentage of untreated control cells.

Apoptosis assessment by annexin V staining: Untreated and drug-treated cells are stained with annexin V and PI, and the percentage of apoptotic cells is determined by flow cytometry. Analysis of synergism between Compound (II) and Compound (V) in inducing apoptosis of K562 and LAMA-84 cells is performed by Median Dose-Effect analysis of Chou and Talalay using the commercially-available software (Calcusyn; Biosoft, Ferguson, Mo.).

Western analyses of proteins: Western analyses are performed using specific antisera or monoclonal antibodies according to previously reported protocols, and the horizontal scanning densitometry is performed on Western blots.

Immunoprecipitation of Bcr-Abl and immunoblot analyses: Following the designated drug treatments, cells are lysed in lysis buffer (20 mM Tris [pH 8], 150 nM sodium chloride, 1% NP40, 0.1 M sodium fluoride, 1 mM PMSF, 1 mM sodium orthovanadate, 2.5 μg/mL leupeptin, 5 μg/mL aprotinin) for 30 minutes on ice, and the nuclear and cellular debris is cleared by centrifugation. Cell lysates (200 μg) are incubated with the Abl-specific monoclonal antibody for 1 hour at 4° C. To this, washed Protein G agarose beads are added and incubated overnight at 4° C. The immunoprecipitates are washed 3 times in the lysis buffer, and proteins are eluted with the SDS sample loading buffer prior to the immunoblot analyses with specific antibodies against anti-Abl or antiphosphotyrosine antibody.

Statistical analysis: Significant differences between values obtained in a population of leukemic cells treated with different experimental conditions are determined using the student t test. P values of less than 0.05 are assigned significance.

Compound (II) and Compound (V) induce apoptosis of K562 and LAMA-84 cells: The effects of Compound (II) and/or Compound (V) in cultured and primary CML-BC cells is determined. The apoptotic effects of treatment with Compound (V) or Compound (II) alone on K562 and LAMA-84 cells is determined. Exposure to Compound (V) or Compound (II) alone induces apoptosis of K562 and LAMA-84 cells in a dose-dependent manner. The data also show that Compound (II) is approximately 10-fold more potent than imatinib in inducing apoptosis of K562 and LAMA-84 cells. Treatment of LAMA-84 cells with Compound (II) inhibited the levels of tyrosine phosphorylated Bcr-Abl in a dose-dependent manner, without affecting the levels of Bcr-Abl. Compound (II) treatment also inhibited the levels of p-CrkL (vide infra), suggesting that AMN107 inhibits the TK activity of Bcr-Abl. Treatment with Compound (II) attenuated the levels of p-STAT5, as well as lowered the expressions of c-Myc and Bcl-xL, which are transactivated by STAT5. Treatment with Compound (II) also inhibited the levels of p-AKT but not AKT, which is associated with induction of p27 levels. This has also been observed following exposure to imatinib. Similar effects of Compound (II) are also observed in K562 cells.

Cotreatment with Compound (V) and Compound (II) exerts superior anti-Bcr-Abl activity and synergistically induces apoptosis of K562 and LAMA-84 cells: Next, the effects of cotreatment with Compound (V) and Compound (II) on Bcr-Abl, as well as on the levels of the signaling proteins downstream of Bcr-Abl is determined. As compared with treatment with either agent alone, relatively low concentrations of Compound (V) (20 nM) and Compound (II) (50 nM) for 24 hours caused more depletion of Bcr-Abl and induced more p27 levels in K562 cells. In contrast, p21 levels are induced to a similar extent by combined treatment with Compound (II) and Compound (V), as compared with treatment with Compound (V) alone. Combined treatment with Compound (V) and Compound (II) also caused more attenuation of the levels of p-CrkL, Bcl-xL, and c-Myc but induced more Bim. Following cotreatment with Compound (II) and Compound (V), simultaneous induction of Bim and attenuation of Bcl-xL is associated with more PARP cleavage, which is due to increased activity of the effector caspases 3 and 7 during apoptosis. Similar effects of Compound (V) and Compound (II) are also observed against LAMA-84 cells. The apoptotic effect of Compound (V) and/or Compound (II) on suspension culture and colony growth of K562 cells is determined. Cotreatment with Compound (II) and Compound (V) caused significantly more inhibition of colony growth than treatment with either drug alone (P<0.05). Similar effect of the combination is also observed against suspension culture growth of K562 cells. The apoptotic effect (increase in the percentage of annexin V-positive cells) of the combined treatment with Compound (II) and Compound (V) in K562 and LAMA-84 cells is determined. Notably, exposure to the combination of Compound (II) and Compound (V) exerted synergistic apoptotic effect in K562 and LAMA-84 cells, as determined by the median dose-effect isobologram analysis. For Compound (II) and Compound (V), the combination index values are less than 1.0 in each cell type. The CI values for K562 are 0.47, 0.36, 0.45, 0.45, and 0.45, respectively, and the CI values for LAMA84 are 0.85, 0.22, 0.21, and 0.16, respectively. The effect of Compound (II) and/or Compound (V) is also determined against NBMCs. Although Compound (II) had no effect (up to 1.0 μM), exposure to 20 and 50 nM Compound (V) for 48 hours induced loss of survival of 13.1% and 15.9% of NBMCs (mean of 2 samples with experiments performed in duplicate). Cotreatment with Compound (II) did not significantly increase the loss of survival of NBMCs because of exposure to 50 nM Compound (V) (P>0.05).

Compound (V) depletes mutant Bcr-Abl levels and induces apoptosis of IM-resistant BaF3 cells expressing Bcr-AblT315I or Bcr-AblE255K: The effect of treatment with Compound (V) and/or Compound (II) on BaF3 cells with ectopic expression of either the unmutated Bcr-Abl or of the point mutant Bcr-AblE255K or Bcr-AblT315I is determined. Similar to the effects seen in K562 and LAMA824 cells with endogenous expression of Bcr-Abl, Compound (II) induced apoptosis of BaF3/Bcr-Abl cells in a dose-dependent manner. Additionally, cotreatment with Compound (II) and Compound (V) induced significantly more apoptosis of BaF3/Bcr-Abl cells than either agent alone. Although exposure to imatinib induced dose-dependent apoptosis of BaF3/Bcr-Abl cells, BaF3/Bcr-AblT315I cells are resistant to imatinib up to levels as high as 10 μM. In contrast, BaF3/Bcr-AblT315I cells are as sensitive as BaF3/Bcr-Abl cells to apoptosis induced by treatment with Compound (V) alone. Treatment with 50 nM Compound (V) for 48 hours induced apoptosis in approximately 30% of BaF3/Bcr-Abl T315I cells. Lower levels of LBH589 are less effective. In contrast, BaF3/Bcr-AblT315I cells are resistant to Compound (II) levels as high as 2,000 nM. Notably, cotreatment with 2,000 nM but not 100 nM Compound (II) significantly increased Compound (V)-induced apoptosis of BaF3/Bcr-AblT315I cells. Against BaF3/Bcr-AblE255K cells, although 100 nM Compound (II) is ineffective, exposure to 200 and 500 nM Compound (II) induced apoptosis of 26.0% and 43.0% of cells, respectively. Again, cotreatment with Compound (II) (500 nM) and Compound (V) (50 nM) induced significantly more apoptosis of BaF3/Bcr-AblE255K cells than treatment with either agent alone (P<0.01), although cotreatment with 100 nM Compound (II) is less effective. Cotreatment with higher concentrations of Compound (II) (1.0 or 2.0 μM) also enhanced Compound (V)-induced apoptosis of BaF3/Bcr-AblE255K. The apoptotic effects of Compound (II) and/or Compound (V) is correlated with their effects on the levels of Bcr-Abl in BaF3/Bcr-Abl, BaF3/Bcr-AblE255K, and BaF3/Bcr-AblT315I cells. Treatment with any of the levels of Compound (II) tested alone did not lower the levels of Bcr-Abl in any of the 3 cell types. Exposure to Compound (II) also did not affect the levels of p-CrkL or CrkL. In contrast, exposure to 50 nM Compound (V) for 24 hours lowered Bcr-Abl levels in all 3 BaF3 transfectants. Notably, as compared with treatment with either agent alone, cotreatment with Compound (V) and Compound (II) induced more depletion of Bcr-Abl in BaF3/Bcr-Abl cells. Notably, combined treatment with Compound (V) and Compound (II) caused a more pronounced decline in the levels of Bcr-AblT315I and Bcr-Abl E255K levels in BaF3/Bcr-AblT315I and BaF3/Bcr-AblE255K cells, respectively. Similar effect is noted on p-CrkL but not CrkL levels.

Cotreatment with Compound (II) and Compound (V) causes more attenuation of Bcr-Abl and loss of viability of primary, imatinib-resistant CML cells than either agent alone: The antileukemia effects of Compound (V) and/or Compound (II) against primary CML cells isolated from the peripheral blood and/or bone marrow samples from 10 patients who had relapsed with Imatinib-resistant CML-BC is determined. Three of these samples are documented to have the expression of Bcr-AblT315I (samples 8, 9, and 10). In the remaining samples of IM-refractory primary CML cells (samples 1 to 7), the mutational status of Bcr-Abl could not be determined, because of inadequate sample size. In the samples 1 to 7 both Compound (II) and Compound (V) induced loss of cell viability, which is dose dependent. Additionally, in these samples, cotreatment with Compound (V) (20 or 50 nM) and Compound (II) induced more loss of cell viability than treatment with either agent alone. Sample 7 is relatively resistant to lower concentrations of Compound (II) but sensitive to Compound (V). In the 3 samples with Bcr-AblT315I mutation (samples 8, 9, and 10), treatment with Compound (II) did not augment loss of cell viability, whereas exposure to Compound (V) alone for 48 hours markedly inhibited cell viability in a dose-dependent manner. Notably, in these samples (8, 9, and 10), cotreatment with 50 or 100 nM Compound (II) did not increase Compound (V)-induced loss of cell viability. In one sample (no. 9), although exposure to even 2.0 μM Compound (II) is ineffective, cotreatment of 50 nM Compound (V) with 2.0 μM Compound (II) induced apoptosis of 63.7% of cells, as compared with apoptosis of 42.0% of cells treated with 50 nM Compound (V) alone. Western blot analyses of the total cell lysates of sample 5 showed that cotreatment with 50 nM Compound (V) and 100 nM Compound (II) for 24 hours resulted in more attenuation of Bcr-Abl, p-CrkL, and p-STAT5 than treatment with either agent alone. In contrast, in sample 9, treatment with even 1000 nM Compound (II) alone had little effect on the levels of Bcr-Abl, p-CrkL, and p-STAT5, whereas cotreatment with 50 nM Compound (V) and Compound (V) markedly depleted the levels of Bcr-AblT315I, as well as of p-CrkL and p-STAT5. These findings are similar to those in BaF3 cells with the ectopic expression of Bcr-AblT315I.

In this example, it is demonstrated that treatment with a combination of the pan-HDAC inhibitor Compound (V) and the Bcr-Abl TK inhibitor Compound (II) synergistically induced apoptosis of cultured mouse pro-B BaF3 and human CML cells with ectopic and endogenous expression of the unmutated Bcr-Abl, respectively. The combination is also more active than either drug alone against BaF3 cells with ectopic expression of the mutant Bcr-AblE255K or Bcr-AblT315I, as well as against imatinib-resistant primary CML cells.

1. A pharmaceutical combination comprising:
a) a pyrimidylaminobenzamide compound of formula (I); and
b) at least one histone deacetylase inhibitor.

2. A pharmaceutical combination according to claim 1, wherein agent a) is selected from 4-methyl-3-[[4-(3-pyridinyl)-2-pyrimidinyl]amino]-N-[5-(4-methyl-1H-imidazol-1-yl)-3-(trifluoromethyl)phenyl]benzamide of formula (II):

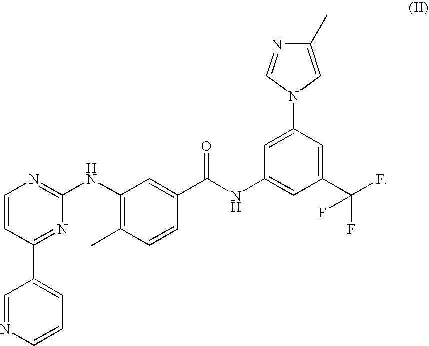

3. A pharmaceutical combination according to claim 2, wherein agent b) is selected from N-hydroxy-3-[4-[(2-hydroxyethyl){2-(1H-indol-3-yl)ethyl]-amino]methyl]phenyl]-2E-2-propenamide, or a pharmaceutically acceptable salt thereof, of formula (IV): 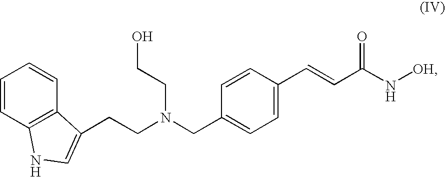
N-hydroxy-3-[4-[[[2-(2-methyl-1H-indol-3-yl)-ethyl]-amino]methyl]phenyl]-2E-2-propenamide, or a pharmaceutically acceptable salt thereof of formula (V) 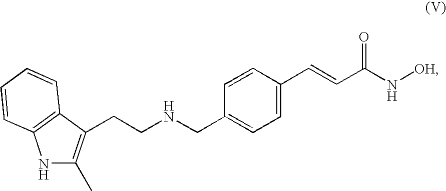
and combinations thereof.

4. A pharmaceutical combination according to claim 2, wherein agent b) is selected from trapoxin, tetrapeptides, chlamydocin, HC Toxin, trichostatin, trichostatin A, apicidin, suberoylanilide hydroxamic acid (SAHA), oxamflatin, MS-275, pyroxamide, valproic acid, [4-(2-amino-phenylcarbamoyl)-benzyl]-carbamic acid pyridine-3-ylmethyl ester and derivatives thereof, Depsipeptide, FR901228, CI-994, phenylbutyrate, sodium butyrate, butyric acid, 3-(4-aroyl-1H-2pyrrolyl-N-hydroxy-propenamides, ADHA compound 8, -(−)Depudecin, Scriptaid, and Sirtinol.

5-7. (canceled)

8. A method for treating or preventing a proliferative disease in a subject in need thereof, comprising co-administration to said subject, e.g., concomitantly or in sequence, of a therapeutically effective amount of at least one histone deacetylase inhibitor and a pyrimidylaminobenzamide compound of formula (I).

9. A method according to claim 8, wherein the disease is a leukemia.

10. A method for treating leukemias comprising administering a combination of an histone deacetylase inhibitor and 4-methyl-3-[[4-(3-pyridinyl)-2-pyrimidinyl]amino]-N-[5-(4-methyl-1H-imidazol-1-yl)-3-(trifluoromethyl)phenyl]benzamide.

11. A method for treating leukemias comprising administering a combination of an histone deacetylase inhibitor and a pyrimidylaminobenzamide compound of formula (I), wherein the histone deacetylase inhibitor is selected from N-hydroxy-3-[4-[(2-hydroxyethyl){2-(1H-indol-3-yl)ethyl]-amino]methyl]phenyl]-2E-2-propenamide, or a pharmaceutically acceptable salt thereof, of formula IV, 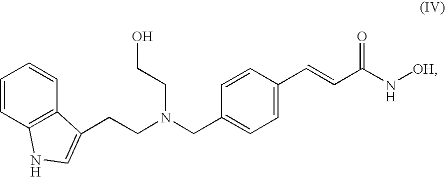
N-hydroxy-3-[4-[[[2-(2-methyl-1H-indol-3-yl)-ethyl]-amino]methyl]phenyl]-2E-2-propenamide, or a pharmaceutically acceptable salt thereof of formula (V) 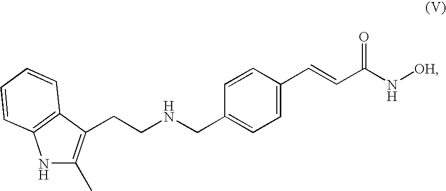
and combinations thereof.

12. A method according to claim 10, wherein the histone deacetylase agent is selected from from trapoxin and other tetrapeptides, e.g., chlamydocin and HC Toxin; trichostatin and its analogues, such as trichostatin A; apicidin; suberoylanilide hydroxamic acid (SAHA); oxamflatin; MS-275; pyroxamide; valproic acid; [4-(2-amino-phenylcarbamoyl)-benzyl]-carbamic acid pyridine-3-ylmethyl ester and derivatives thereof; Depsipeptide FR901228; CI-994; phenylbutyrate; sodium butyrate; butyric acid; 3-(4-aroyl-1H-2pyrrolyl-N-hydroxy-propenamides; ADHA compound 8; -(−)Depudecin; Scriptaid; and Sirtinol.

13. A method according to claim 8, wherein the disease is chronic myelogenous leukemia or acute lymphocyte leukemia.
